(19) United States
(12) Patent Application Publication
(10) Pub. No.: US 2022/0036991 A1
(43) Pub. Date: Feb. 3, 2022

(54) SYSTEM FOR TRACKING A HEALTH-RELATED BEHAVIOR

(71) Applicant: AARDEX GROUP, Seraing (BE)

(72) Inventors: Nicolas TILLE, Seraing (BE); David DALLA VECCHIA, Seraing (BE); Bernard VRIJENS, Seraing (BE)

(21) Appl. No.: 17/276,467

(22) PCT Filed: Sep. 27, 2019
(86) PCT No.: PCT/EP2019/076208

§ 371 Date: Mar. 15, 2021

(30) Foreign Application Priority Data
Oct. 1, 2018 (BE) 2018/5670

(51) Int. Cl. G16H 20/13; G16H 40/67; A61J 7/04

(52) U.S. Cl. CPC G16H 20/13; G16H 40/67; A61J 2200/70; A61J 2200/30; A61J 7/0418

(57) ABSTRACT

The present invention relates to a system for tracking a health-related behaviour, comprising a support and an electronic surveillance device having a casing, from which a first wall extends, a first portion of said wall extending from the casing in a plane that intersects the casing and being connected to the casing, and a second portion of said wall extending in a plane parallel to the casing, being connected by an end linked to the first portion and being provided with a second free end, wherein the first and second portions together form a slide arranged to cooperate with a wall of the support.

The present invention relates to a system for tracking a health-related behaviour, and more particularly to a system for tracking a health-related behaviour comprising a support and an electronic monitoring device as well as to the use thereof for measuring and managing a habit such as for example a patient's compliance with a prescribed dosage.

The term “tracking a health-related behaviour” means, in the sense of the present invention, measuring and managing the administration of a medical treatment involving a medication or several medications combined, a therapy recommended by a health professional (treatments with light against depression, PUVA therapies against skin diseases, or allergy desensitization treatments), and generally any habit connected with a health-related behaviour that we wish to initiate (drinking a glass of water at repeated intervals, etc.) or modify (smoking, access to the larder, biting one's nails, etc.).

“Medicinal product/medication” means, in the sense of the present invention, a substance or a composition containing one or more active ingredients presented as possessing therapeutic or preventive properties.

A patient's compliance with a dosage regimen of a medicinal product is of increasing interest nowadays. The dosage regimen, also called posology, of a medicinal product prescribed for a patient is defined both by the recommended dose each time it is taken, and by the frequency with which it is taken. Knowing that when the medicinal product is taken, the active ingredient or ingredients is/are metabolized and absorbed by the patient, its plasma concentration then increases and decreases over time. The dosage regimen aims to maintain the plasma concentrations of the active ingredient or ingredients within the therapeutic range, i.e. in the range of concentrations thereof ensuring the optimum therapeutic or preventive or prophylactic effect of active ingredient(s) while minimizing the risk of undesirable effects. It aims not to go below the minimum concentration corresponding to under-dosage for the patient, which would lead to an increased risk of failure of therapy or prevention, and aims not to exceed a maximum concentration of active ingredients for the patient, which would correspond to an overdose, which would lead to a higher risk of toxicity. In this context, treatments with delayed or sustained release have been developed.

The frequency with which the medicinal product is taken by the patient, and therefore adherence to the dosage regimen that has been prescribed, depends on many factors, intentional or otherwise (forgotten). Moreover, if side-effects occur, the patient's reticence to take the prescribed dose has the result that he may delay one or other dose for reasons of comfort, in order to suffer the side-effects in a place or at a time that is more suitable than another.

Therefore systems comprising electronic devices have been developed for tracking the taking of medicinal products.

For example, packages for medicinal products comprising an electronic device are known from the prior art, such as those used for measuring the precise time that the medicinal product is taken by the patient, which can be compared against the prescribed dosage regimen. This measurement of the variation between behaviour expected of the patient and the measured behaviour can provide health professionals with better monitoring of their patients. Said packaging also sometimes has functionalities for reminding the patient to take the medicine. Furthermore, it is possible to use an electronic device pre-programmed according to the posology prescribed by the doctor (“reminder” device); this electronic device emits an alarm signal based on sound or light or else via a smartphone application for reminding the patient that he has to take his treatment.

However, often the device, for example such as a smartphone, might well be in one place, for example such as in a living room, in a bag, or in a suitcase, whereas the medication is elsewhere.

Such packaging for medicinal products comprising an electronic device is used in particular when a medicinal product is to be taken at irregular frequencies, or several medicinal products are to be taken at different frequencies, or for medicinal products especially where under-dosage or overdosage may have important consequences for the patient's health, as well as for patients who forget to take their treatments.

Even in the context of treatments with a narrow therapeutic window, academic studies or clinical studies, the patients being treated often deviate from the prescribed dosage regimen of the medicinal product. In the case of treatment with a narrow therapeutic window, for example such as antirejection treatments, or chemotherapy, it is important to take the medicine at regular intervals, which implies remembering when it was last taken, as the regularity of taking the doses is a key factor in the success of the treatment.

Regularity is also particularly important in the case of clinical studies.

In both cases, whether it is a question of medical practice or of clinical studies, it is particularly important to be able to identify the causal link between taking, forgetting to take or irregularity of taking medicinal products and the success of a treatment or the generation of side-effects or else non-efficacy of a treatment. The causal link identified makes it possible to adapt the patient's posology in the case of treatments or to adapt the posology to be prescribed when the medicinal product in the study is to be put on the market or to act in order to modify the patient's behaviour. Consequently, measurement of a patient's compliance with a treatment has increasingly become a centre of interest.

In the context of academic studies or clinical studies relating to drug development, measurement of deviations from the prescribed dosage regimen is the main objective of the electronic device for monitoring the taking of medication, and with regard to the induced functionality of reminding, it is often secondary or even undesirable so as not to introduce a bias in the measurement of efficacy of the medicinal product. Several developments have therefore been carried out for perfecting electronic devices so that the latter record the moment, for example the date and time (or a time interval) of taking medication.

Thus, the data collected by the electronic device corresponding to the moment, such as the time and the date, of each taking of medication make it possible to evaluate the efficacy and tolerance of a treatment more precisely, but also to inform the health professional in the case when a patient has not adhered to the prescription given, which could have an effect on his state of health.

Thus, measurement of the patient's adherence to the prescribed treatment may allow the health professional to evaluate whether the posology can be adapted case by case. In fact, the health professional may for example decrease the dosage of the medicinal product or else give a wider interval between the individual doses of the medicine in order to make it more comfortable for the patient.

The information on taking medication such as the date and time therefore result either from a deliberate act on the part of the patient connected with his goodwill when the latter will signal the taking of medication with an electronic monitoring device with mechanical or electronic operation, or from a sensor with passive operation in the electronic device which records an event, the opening and closing of packaging for medicinal products, and which therefore is not connected with a deliberate act on the part of the patient.

For example, a system as described in document WO 01/08106 is known from the prior art.

Document WO 01/08106 discloses a system for tracking a health-related behaviour comprising:
a support, and
an electronic monitoring device for generating data indicating a health-related behaviour based on capture of events comprising a casing in which there is a power source and a processor, on which there is optionally a display device and from which a first wall extends, a first part of which extends from said casing in an intersecting plane relative to the casing and is connected to said casing and a second part, which extends in a plane parallel to the casing, connected by one end joined to the first part and provided with a second free end.

Two embodiments are described in this document.

According to the first embodiment, the packaging for medicinal products comprises an integrated circuit chip fixed directly to the packaging, for example a blister, a bottle or a tube. According to the second embodiment, an integrated circuit chip is fixed on an additional support of the packaging for medicinal products.

In all cases, the integrated circuit chip is inseparable from the packaging for medicinal products, since it contains information on the medicinal product in question for example such as recommendations relating to taking, incompatibilities or else corrective measures in case of non-compliance with the recommendations for taking the medication. Thus, the posology or dosage is information stored on the integrated circuit chip and therefore integral with a standard medication.

The aim pursued by the system in document WO 01/08106 is to encode this information in the memory of the chip during packaging of the medicinal products, before making the treatment available to the patient.

Furthermore, the system in document WO 01/08106 supplies an electronic monitoring device that comprises a slide comprising two slits. In the first embodiment, the slide interacts with the chip contained in the packaging by means of two edges (blister, bottle) which will fit into the slits of the slide provided for this purpose or without contact (tube). In the second embodiment, the slide interacts with an additional support of the packaging for medicinal products, said additional support comprising the integrated circuit chip. The additional support is therefore inseparable from the packaging for medicinal products since it allows good positioning of the electronic monitoring device against the integrated circuit chip, so that contact occurs.

In fact, in operation, in the first embodiment, the patient will be provided with the electronic monitoring device and will affix it directly on the integrated circuit chip of the blister, bottle or tube. In the second embodiment, the patient will fit the slide in the space provided for this purpose by the additional support of the packaging.

In both cases, the patient will bring the integrated circuit chip and the electronic monitoring device into contact with one another.

The electronic monitoring device then performs the role of an interface that allows the user to visualize the data stored on the integrated circuit chip. It appears that the integrated circuit chip is inseparable from the packaging for medicinal products, as it is the chip that contains and stores the data, for example when the patient will perform the action of taking the medication. In fact, the data indicating the taking of medication will, by means of the clock of the integrated circuit chip and of the processor of the electronic monitoring device, be stored in the chip's memory so that the health professional can collect the medicine packaging and have access to the data directly from the integrated circuit chip.

Unfortunately, the system for tracking a health-related behaviour according to that document is totally dependent on its support, which contains the chip, fixed beforehand, directly or via an additional support. Fixation of the chip takes place before being made available to the patient, which makes the industrial execution of the packaging for medicinal products expensive, and it does not allow the electronic monitoring device according to that document to be adaptable to a large number of conventional supports. It is also necessary to avoid discarding the packaging as it contains information.

There is therefore a general need for a system for tracking a health-related behaviour that is adaptable and reliable but is easy to manufacture and implement.

The aim of the invention is to solve the aforementioned problems by providing a system for tracking a health-related behaviour as mentioned at the beginning, characterized in that said first and second parts together form a slide configured to interact with a wall of said support, and in that said device comprises a memory and a clock, both integrated in said casing.

According to the present invention, the system is very adaptable, since the slide can interact with any type of support, which can be held tightly between the second part of the wall that extends from the casing of the monitoring device and the casing itself. The casing of the adaptable system is generally placed outside of the support and contains the memory and the clock, which facilitates programming of the electronic monitoring device and allows it to be produced separately from the manufacture and packaging of the medicinal product. Discarding of the information is avoided, as it is in the casing of the electronic monitoring device and not in the packaging.

Accordingly, the electronic monitoring system according to the present invention offers an alternative form of tracking that differs from passive, forced capture associated with the event sensors fixed and integrated in the packaging, such as those which capture the opening of the packaging. Although passive systems are of some benefit in particular applications, they do not provide confirmation that the medication has actually been taken, since it is the opening of the box that is captured. They are also dependent on the packaging and must be integrated generally, all or part during manufacture.

The system according to the present invention also offers an alternative to the applications integrated with smartphones, which also present certain disadvantages such as being unsuitable for the elderly, lack of potential proximity between the smartphone and the treatment, the need to manage data protection over the internet, etc.

Consequently, the availability of an electronic monitoring device separate from the packaging and outside the latter, adaptable to many supports while being provided with a memory and a clock, makes it possible to combine the advantages of several monitoring systems while eliminating their drawbacks.

According to the present invention, the system is in fact very flexible as it can be adapted to many supports, but is also programmed separately from manufacture while being very reliable both by the preservation of data that are not necessarily transferred and certainly not in an uncontrolled manner via the internet and allowing capture of the real health-related behaviour on a voluntary basis, for example the actual taking of the medicine and not just the opening of the packaging.

This combination of adaptability and reliability is linked intrinsically to the questioning of the fundamentals on which the existing tracking systems are based, by becoming free on the one hand from the constraints of forced integration of the passive capture devices and on the other hand by becoming free from the difficulty connected with the non-proximity of smartphones and associated with management of data security. This is achieved with an autonomous casing that comprises its own fixation system but also its own processor, clock and memory.

Consequently, the events of a health-related behaviour will be captured, for example on a voluntary basis, in the electronic monitoring device via the autonomous casing, fixed by its slide to the support of choice, for example such as a medicine box, the fabric of a pocket of a garment, a wallet, a bag, in all cases a support within easy reach for the patient. The events will be processed directly in the casing by the processor and, owing to the presence of the clock in the casing, the processor will generate data indicative of the health-related behaviour corresponding to the events of the health-related behaviour captured, and record them in the memory, directly present in the casing of the electronic monitoring device.

The patient but also the health professionals are thus provided with an autonomous system that is versatile and adaptable to a wide range of supports and whose electronic monitoring device is easy to use, reliable in terms of data capture, and manipulation of which, for example fixing or moving, is easy for the patient.

In a preferred embodiment according to the present invention, said electronic monitoring device comprises a sensor configured for capturing an event of a health-related behaviour, preferably said sensor is a passive sensor, mechanical for example a push-button, physical, electronic, electromagnetic, luminous, chemical, preferably a sensor with low energy consumption.

In fact, the adaptable, autonomous electronic monitoring device may integrate different types of sensors, which capture the events introduced voluntarily but also passively if necessary.

In a preferred embodiment, the sensor is configured for capturing an event of a health-related behaviour on a voluntary basis on the part of the patient, which is particularly important in the context of measurement of the patient's voluntary compliance.

Moreover, this makes it possible to move the electronic monitoring device from one support to another support and, in the case when the support is the packaging of the medicinal product, so that the electronic monitoring device with a sensor according to the present invention is moved from one medicine package to another medicine package when the medicines are used up. Thus, this makes it possible to reuse the electronic system on a new package for a time of up to three years, but also avoids the battery becoming empty during treatment or if the electronic monitoring device is stored for several months, especially when the sensor is a low-energy sensor.

Preferably, according to the present invention, the casing has a wall facing the second part of the wall of the slide, and at least one of either the wall or the slide comprises means for securing the electronic monitoring device on the support.

Advantageously, according to the present invention, said second part of the wall of said slide comprises a first face and a second face parallel to the first face, said first face being exposed to the exterior and said second face being exposed towards the casing, and is provided with a lug that extends from the second face to the casing.

Preferably, according to the present invention, the second part of the wall of said slide is provided with a retaining means, preferably a lug, which extends from the second face to the casing.

In fact, the presence of a retaining means, preferably at least one lug on the second part of the wall of the slide exposed to the casing, makes it possible to perform a first squeezing of a wall of a support, for example of the medicine package at the time of fixing the electronic monitoring device. This makes it possible in particular to fix the electronic monitoring device firmly to the support or to the medicine package, so that the electronic monitoring device does not become detached from the medicine package in the case of impact or if it is dropped. Thus, this first squeezing is reinforced by the slight elasticity of the material of the slide.

Advantageously, according to the present invention, said retaining means further comprise a lug extending from said wall in the direction of the second part of the wall of the slide, preferably under the slide.

In a variant, according to the present invention, said casing has a wall facing said second part of the wall of said slide comprising a lug extending from said wall in the direction of the second part of the wall of the slide, preferably under the slide.

In fact, the presence of at least one lug on the casing of the electronic monitoring device extending in the direction of the slide makes it possible to perform a second squeezing of a wall of the support or of the medicine package at the time of fixing the electronic monitoring device. This makes it possible in particular to perform optimum squeezing for fixing the electronic monitoring device firmly to the medicine package. Thus, this second squeezing is reinforced by the slight elasticity of the material of the slide.

Advantageously, the electronic monitoring device comprises a first lug extending from the second face of the slide to the casing and a second lug that extends from the wall of the casing to the second part of the wall of the slide.

In fact, the first lug and the second lug allow double squeezing of a wall of the support or of the medicine package or of any other support that can be held tightly at the time of fixing the electronic monitoring device. This makes it possible in particular to achieve optimum squeezing for fixing the electronic monitoring device firmly to the medicine package, all the more so because this double squeezing is reinforced by the slight elasticity of the material of the slide.

In a preferred embodiment, the support of the system according to the present invention is a socket.

In another preferred embodiment according to the present invention, said socket is configured for being fixed permanently or temporarily to a base, said socket being provided with a wall comprising a lower face and an upper face from which two guide rails extend, with each extending in a longitudinal direction parallel to the upper face of said socket, each rail being a sectional bar provided with a first wall extending perpendicularly to said upper face of said socket, along the latter, and a second wall extending approximately perpendicularly from said first wall and parallel to said upper face of said socket, said socket being configured for housing the electronic monitoring device.

According to the present invention, each rail is a sectional bar provided with a first wall extending perpendicularly to said upper face of the socket, along the latter, and with a second wall extending approximately perpendicularly to said first wall and parallel to said upper face of the socket, which makes it possible to provide a socket on which two rails extend, of a shape that is easy to manufacture on a large scale by injection moulding without the socket and its rails being deformed during mould release.

Furthermore, by providing a socket configured for being fixed to a support and configured for housing the electronic monitoring device, the present invention allows the electronic monitoring device to be fixed on a very wide range of bases. In fact, the base may be a medicine box, a tablet container, a weekly dispenser, a bottle, but also a garment, a piece of furniture, a sanitary fitting, etc.

The electronic monitoring device can easily be housed in the socket, itself fixed firmly to a base, which allows optimum fixing so that the electronic monitoring device does not become detached from the socket, nor from the base in the case of impact or if dropped. The electronic monitoring device is advantageously detachable in order to be moved from one base to another when the patient has exhausted the medicines of a medicine box or a bottle for example.

Furthermore, according to the present invention, each wall of the aforementioned two guide rails comprises retaining means, for example at least one lateral projection.

According to the present invention, said slide comprises a first lateral face and a second lateral face, each lateral face comprising retaining means, for example at least one slot. Advantageously, according to the present invention, each first wall of said two guide rails comprises at least one lateral projection, preferably opposite one another, and said second part of the wall of said slide comprises a first lateral face and a second lateral face, each lateral face comprising at least one slot, said slots being located on either side of said second part of said slide and being configured for housing said lateral projections of said socket, when the casing of the electronic monitoring device is housed in the socket, preferably by forced fitting.

According to the present invention, the fact that each first wall of the guide rails comprises at least one lateral projection and that each lateral face of the slide comprises at least one slot allows the electronic monitoring device to be housed via its slide in the socket precisely and optimally, preferably by forced fitting.

In fact, a system according to the present invention, comprising an electronic monitoring device housed in a socket, itself fixed to a base, advantageously allows precise, fixed positioning of the electronic monitoring device. Thus, insertion and withdrawal of the electronic monitoring device via the slide in the socket are easy to perform by a translational motion. The electronic monitoring device in the inserted position is blocked, thus preventing separation in the case of impact or if dropped. Furthermore, manufacture and implementation are easy.

In a variant according to the present invention, said base may be a medicine box, a tablet container, a weekly dispenser, a bottle, a garment, a piece of furniture, a sanitary fitting.

Advantageously, according to the present invention, said lower face of said socket comprises an adhesive, for example of the double-sided type, self-gripping means, for example a Velcro® fixing, or a repositionable pressure-sensitive silicone.

In fact, with an adhesive, according to a first embodiment, the socket can easily be fixed to its support and be moved from one support to another, for example when the patient has used up all the medicine. Preferably, according to a second embodiment, the lower face of the socket comprises a strong adhesive. This allows the patient to fix the socket firmly to a support, for example a sanitary fitting, with the electronic monitoring device housed in the socket; the patient can then capture a health-related behaviour, for example drinking a glass of water.

In the context of the present invention, the term “retaining means” denotes retaining means selected from the group comprising at least one lug that extends from the second face of the second part of the wall of the slide to the casing, at least one lug extending from said wall of the casing in the direction of the second part of the wall of the slide, at least one lateral projection of each first wall of said two guide rails, at least one slot of each lateral face of said slide on either side of said second part or else any other means allowing the electronic monitoring device to be engaged and disengaged, and a combination thereof. For example, in the case of guide rails that are parallel and concentric, the retaining means can engage and disengage the electronic monitoring device by simple rotation or some other movement of fitting, preferably by forced fitting.

Other embodiments of the system for tracking a health-related behaviour are presented in the accompanying claims.

The present invention also relates to the use of the system for tracking a health-related behaviour according to the present invention, for measuring a patient's compliance with a prescribed dosage.

It was in fact found that having the system for tracking a health-related behaviour according to the present invention is advantageous for measuring the patient's adherence to a prescribed dosage.

Other embodiments of the use of the system for tracking a health-related behaviour are presented in the accompanying claims.

The invention will now be described in more detail, referring to the appended figures.

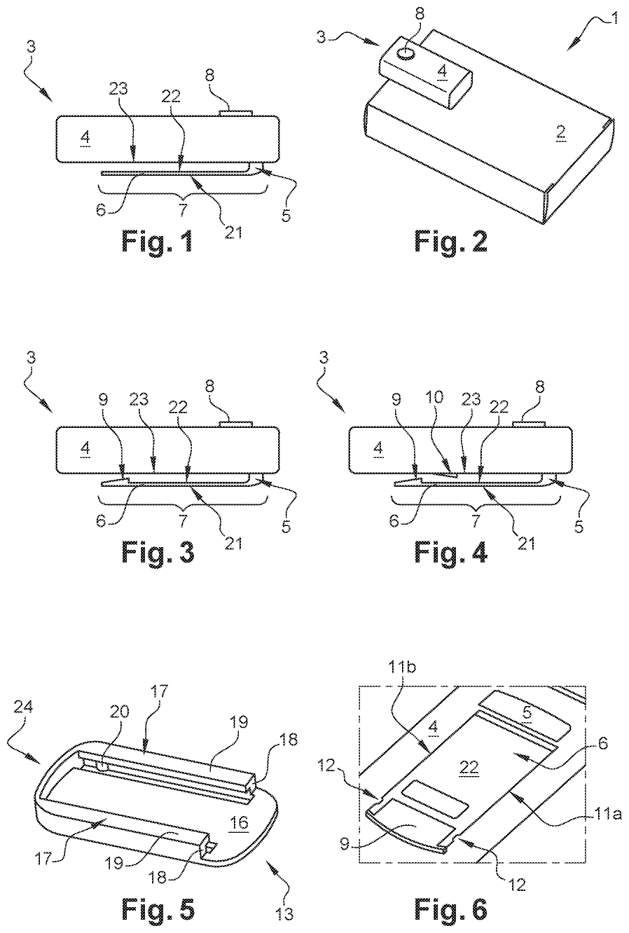

FIG. 1 shows a side view of the electronic monitoring device according to the present invention.

FIG. 2 shows a top view of the system according to the present invention showing the electronic monitoring device and a support.

FIGS. 3 and 4 show a side view of two alternatives of the electronic monitoring device according to the present invention.

FIG. 5 shows a perspective view of the socket according to the present invention.

FIG. 6 shows a sectional view of the slide of the electronic monitoring device according to the present invention.

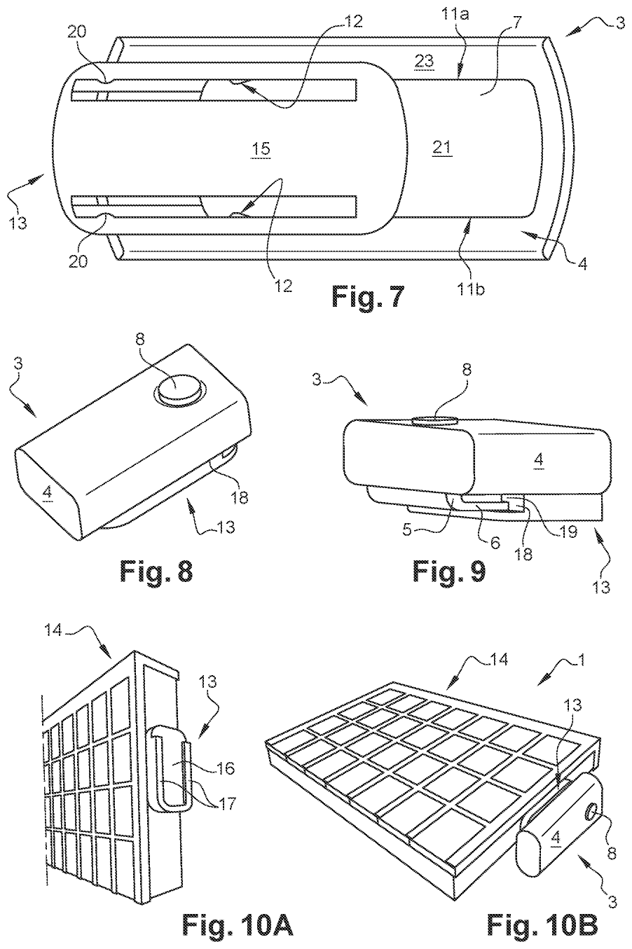

FIG. 7 shows a bottom view of the fixing of the electronic monitoring device on the socket according to the present invention.

FIG. 8 shows a perspective view of the electronic monitoring device fixed to a socket according to the present invention.

FIG. 9 shows an alternative perspective view of the electronic monitoring device fixed to a socket according to the present invention.

FIGS. 10A and 10B show a perspective view of the system according to the present invention showing the base, the socket and the electronic monitoring device.

In the figures, elements that are identical or similar bear the same reference numbers. FIG. 1 illustrates an electronic monitoring device 3 that makes it possible to generate data indicative of the health-related behaviour based on capture of events.

According to the present invention, the electronic monitoring device 3 comprises a casing 4 in which there are a power source, a processor, a sensor, a clock and a memory.

The power source is for example a battery or a cell and is configured for supplying an electric circuit connecting together the different elements present in the casing 4.

The processor is connected to the power source, the sensor, the clock and the memory and is configured for processing an event connected with a health-related behaviour and for generating a signal comprising the data indicative of the health-related behaviour.

In a preferred embodiment, the casing 4 of the electronic monitoring device 3 comprises a sensor 8 configured for capturing an event of a health-related behaviour, preferably the sensor 8 is a passive sensor, mechanical for example a push-button, physical, electronic, electromagnetic, luminous, chemical, preferably a sensor with low energy consumption.

The sensor 8, preferably a push-button, acts as a switch with normally open operation. In fact, electrical connection is created when the patient presses the push-button.

In operation, when the patient performs a health-related behaviour, for example such as taking a medical treatment or monitoring a physical measurement, he will actuate the sensor 8, which will create an electrical connection between the power source and the processor. The processor receives the event of the health-related behaviour by the creation of the electrical connection and will then process this event. The processor is configured for sending a signal to the clock for recovering the data on time and date contained in the clock. After receiving the data on time and date from the clock, the processor generates data indicative of the health-related behaviour based on event capture and sends a signal to the memory, more precisely the processor records the data indicative of the health-related behaviour in the memory.

Optionally, the casing 4 of the electronic monitoring device 3 comprises an indicator lamp, an audible device, or a vibrator for indicating the event of the health-related behaviour. According to one embodiment, the indicator lamp, for example an LED, the audible device or the vibrator is located before the processor in the electric circuit, more particularly between the sensor and the processor. In this first embodiment, when the patient actuates the sensor 8 and creates the electrical connection, the indicator lamp, the audible device or the vibrator is supplied, which leads to illumination of the indicator lamp, emission of a sound from the audible device or a vibration to indicate to the patient that the sensor 8 has been actuated correctly. According to another embodiment, the indicator lamp, the audible device or the vibrator is located after the processor in the electric circuit. In this second embodiment, when the patient actuates the sensor 8 and creates the electrical connection, the processor processes the event of the health-related behaviour as described above and then the indicator lamp, the audible device or the vibrator is supplied, which leads to illumination of the indicator lamp, emission of a sound from the audible device or a vibration to indicate that the sensor 8 has been actuated correctly and that the event of the health-related behaviour has been processed correctly by the processor. In another embodiment, the indicator lamp, the audible device or the vibrator may be integrated with the sensor 8 directly.

In all cases, these elements aim to improve the behaviour related to the patient's health, more particularly they aim to improve the patient's compliance behaviour.

In another embodiment, the casing 4 of the electronic monitoring device 3 is provided with a display device, preferably an LCD screen on its upper face, as opposed to the lower face 23, which is in contact with the wall of the support 2. The display device is configured for communicating visual information to the patient. In operation, the processor sends a signal to the display device. This signal is translated by the display device into visual information that can be read by the patient. The visual information is for example an indication of the number of doses taken recorded by the patient, the time remaining before the next event of the health-related behaviour or else the degree, as a percentage, of good or poor compliance behaviour of the patient. The display device is configured to be put back to zero at intervals of 24 hours.

Furthermore, these elements (indicator lamp, audible device, vibrator or display device etc.) may be used for supplying information to the patient. For example, an LED may be programmed to light up or emit flashes for two hours after an event connected with a health-related behaviour and then stop, to indicate that the event must be renewed, or vice versa. Thus, the audible device or the vibrator can be programmed to emit an audible signal or a vibration, respectively, two hours, four hours, six hours, eight hours etc. after the event of the health-related behaviour, for example taking a medical treatment, to signal to the patient that the event must be renewed.

Optionally, the system 1 for tracking a health-related behaviour comprises an external reader configured for recovering the data indicative of the health-related behaviour recorded in the memory by the processor. Preferably, the external reader is a remote reader, operating by various known methods such as NFC technology, Bluetooth, Wi-Fi, infrared etc.

Furthermore, the casing 4 of the electronic monitoring device 3 comprises all the elements necessary for capturing an event of a health-related behaviour, for generating data indicative of the health-related behaviour based on event capture and therefore for example for measuring a patient's voluntary adherence.

Furthermore, a first wall extends from the casing 4 of the electronic monitoring device 3. A first part 5 of said first wall extends from the casing 4 in a plane intersecting the casing 4 and joined to the casing 4. A second part 6 of said first wall extends in a plane parallel to the casing 4, is connected by one end joined to the first part 5 and is provided with a second free end. Said first part 5 and said second part 6 of the first wall extending from the casing 4 together form a slide 7, which interacts with a wall of the support 2, for example a wall of a medicine package.

In fact, the slide 7 can interact with any type of supports 2 but also with other bases 14 which can be held tightly between the second part 6 of the first wall that extends from the casing 4, and the casing 4 itself.

Thus, the slide 7 of the casing 4 of the electronic monitoring device 3 comprises a first face 21 and a second face 22 parallel to the first face 21. The first face 21 is exposed to the exterior, and the second face 22 is exposed to the casing 4.

FIG. 2 illustrates the system 1 for tracking a health-related behaviour according to the present invention, which comprises a support 2 and an electronic monitoring device 3 for generating data indicating a health-related behaviour based on capture of events.

The slide 7 of the casing 4 of the electronic monitoring device 3 is configured to interact with a wall of the support 2. In fact, the first part 5 extending from the casing 4 and the second part 6, together forming the slide 7, allow a space to be created between the slide 7 as such and the casing 4, so that the user can position the electronic monitoring device 3 as shown in FIG. 2, the wall of the support 2 being introduced into the space between the slide 7 and the casing 4 until it comes up against the first part 5.

FIG. 3 illustrates an alternative of the electronic monitoring device 3 according to the present invention. The slide 7 of the casing 4 of the electronic monitoring device 3 comprises a first face 21 and a second face 22 parallel to the first face 21. The first face 21 is exposed to the exterior, and the second face 22 is exposed to the casing 4. The slide 7 is provided with a lug 9 that extends from the second face 22 to the casing 4.

The presence of at least one lug 9 on the slide 7, which extends from the second face 22 to the casing 4, makes it possible to fix the electronic monitoring device 3 on the support 2, for example a wall of a medicine package, but also with other bases 14, by a first squeezing so that fixation is reinforced. This fixation by squeezing makes it possible to prevent the electronic monitoring device 3 becoming detached from the support 2 in the case of impact or if dropped.

FIG. 4 illustrates another alternative of the electronic monitoring device 3 according to the present invention. The casing 4 of the electronic monitoring device 3 has a wall 23 that faces the second part 6 of the wall of the slide 7 and that comprises a lug 10 that extends from said wall 23 to the second part 6 of the wall of the slide 7, preferably under the slide 7.

In fact, the presence of at least one lug 10 on the casing 4 that extends from a wall of the casing 4 to the second part 6 of the wall of the slide 7 makes it possible to fix the electronic monitoring device 3 on the support 2, for example the wall of a medicine package, but also with other bases 14, by a second squeezing so that fixation is reinforced. This fixation by squeezing makes it possible to prevent the electronic monitoring device 3 becoming detached from the support 2 in the case of impact or if dropped.

Advantageously, the electronic monitoring device 3 comprises a first lug 9 that extends from the second face 22 of the slide 7 to the casing 4 and a second lug 10 that extends from the wall 23 of the casing 4 to the second part 6 of the wall of the slide 7. In this embodiment, the first lug 9 and the second lug 10, at the time of fixing the electronic monitoring device 3 to a support 2 but also to other bases 14, allow double squeezing. In fact, fixation by double squeezing allows the electronic monitoring device 3 to be made integral with the support 2 so that it remains correctly positioned and fixed during an impact or if dropped.

FIG. 5 illustrates the socket 13 according to the present invention. The socket 13 is provided with a wall that comprises a lower face 15 (not visible) and an upper face 16, from which two guide rails 17 extend. The two guide rails 17 each extend in a longitudinal direction parallel to the upper face 16 of the socket 13. Each rail 17 is a sectional bar provided with a first wall 18 extending perpendicularly to the upper face 16 of the socket 13, along the latter, and a second wall 19 that extends approximately perpendicularly to the first wall 18 and parallel to the upper face 16 of the socket 13. Furthermore, the socket 13 also comprises a stop element 24, serving to prevent insertion of the electronic monitoring device 3 the wrong way round.

According to the present invention, each first wall 18 of the guide rails 17 comprises at least one lateral projection 20, preferably the at least one lateral projection 20 of each guide rail 18 are opposite one another.

FIG. 6 illustrates a sectional view of the slide 7 according to the present invention. The slide 7 comprises a first part 5 extending from the casing 4, a second part 6 and a lug 9, as described above.

According to the present invention, the second part 6 of the wall of the slide 7 comprises a first lateral face 11a and a second lateral face 11b; each lateral face comprises at least one slot 12. The slots 12 are located on either side of the second part 6 of the wall of the slide 7 and are configured for housing the lateral projections 20 of the guide rails 17 of the socket 13, when the electronic monitoring device 3 is housed in the socket 13, preferably by forced fitting.

FIG. 7 shows a bottom view of the fixation of the electronic monitoring device 3 in the socket 13. According to the present invention, it can be seen that the two slots 12 of the slide 7 of the electronic monitoring device 3 and the two lateral projections 20 of the guide rails 17 of the socket 13 are of complementary shape and are able to interact by forced fitting.

Furthermore, the lateral projections 20 are of complementary shape with that of the slots 12, in such a way that when the patient wishes to fix the electronic monitoring device 3 in the socket 13, which in its turn is positioned on its base 14, the patient only has to insert the slide 7 in the space provided for this purpose, between the upper face 16 of the socket 13 and the second wall 19 of the guide rails 17 and push the electronic monitoring device 3 in a translational motion until there is forced fitting of the lateral projections 20 in the slots 12 provided for this purpose.

According to the present invention, the electronic monitoring device 3 in the mounted state, when it is positioned in the socket 13, is blocked and immobile in the three spatial directions so that it cannot be displaced in the case of impact or if dropped.

In another embodiment, each first wall 18 of the guide rails 17 may comprise at least one lateral slot, preferably the at least one lateral slots of each guide rails 17 are opposite one another, and each lateral face of the second part 6 of the wall of the slide 7 may comprise at least one projection, preferably the projections are located on either side of the second part 6 of the wall of the slide 7.

Furthermore, the lower face 15 of the socket 13 according to the present invention comprises an adhesive, for example of the double-sided type, self-gripping means, for example a Velcro® fixing, a repositionable pressure-sensitive silicone, in order to be able to fix the socket 13 easily to a base 14 and reposition the socket 13 easily on its base 14 or on another base 14 when this is necessary, for example when the patient wishes to change the weekly dispenser, tablet container or when the patient has used up all the medicine.

Alternatively, the socket 13 according to the present invention may comprise a system for fixing by screw or by rivet, to be fixed to a base 14.

FIG. 8 illustrates fixation of the electronic monitoring device 3 to a socket 13. The slide 7 of the casing 4 of the electronic monitoring device 3 is configured for being inserted between the rails 17 of the socket 13.

FIG. 9 illustrates another view of the fixation of the electronic monitoring device 3 to the socket 13. In fact, the slide 7 of the casing 4 will be housed in a space created between the second wall 19 of the guide rails 17 and the upper face 16 of the socket 13.

FIG. 10A illustrates fixation of the socket 13 on a base 14, for example a weekly dispenser.

FIG. 10B illustrates the system 1 according to the present invention comprising a base 14, for example a weekly dispenser, on which the socket 13 is fixed, into which the electronic monitoring device 3 will be inserted for tracking the health-related behaviour of a patient and more particularly for measuring the patient's compliance.

Furthermore, the base 14 may be a medicine box, a tablet container, a weekly dispenser, a bottle, a garment, a piece of furniture, a sanitary fitting or any other object to which the patient has access.

According to the present invention, the system 1 for tracking a health-related behaviour is used for measuring a patient's compliance with a prescribed dosage.

Of course, the present invention is not in any way limited to the embodiments described above and many modifications may be made while remaining within the scope of the accompanying claims.

Although the electronic monitoring device, its casing and its support have been defined with reference to guide rails each extending in a parallel longitudinal direction, it goes without saying that the invention also protects parallel and concentric guide rails provided with retaining means that make it possible to keep the electronic monitoring device engaged and to disengage it for example by simple rotation or some other movement.

1. A system for tracking a health-related behaviour comprising:
a support, and
an electronic monitoring device for generating data indicating a health-related behaviour based on capture of events comprising a casing in which there is a power source and a processor, on which there is optionally a display device and from which a first wall extends, a first part of which extends from said casing in a plane intersecting the casing and connected to said casing and a second part that extends in a plane parallel to the casing, connected by one end joined to the first part and provided with a second free end,
characterized in that said first part and second part together form a slide configured to interact with a wall of said support, and in that said device comprises a memory and a clock, both integrated in said casing.

2. The system according to claim 1, in which said casing has a wall facing said second part of the wall of said slide and at least one of either said wall or said slide comprising means for retaining said electronic monitoring device on said support.

3. The system according to claim r 2, in which said electronic monitoring device comprises a sensor configured for capturing an event of a health-related behaviour, preferably said sensor is a passive sensor, mechanical for example a push-button, physical, electronic, electromagnetic, luminous, chemical, preferably a sensor with low energy consumption.

4. The system according to claim 2, in which the second part of the wall of said slide comprises a first face and a second face parallel to the first face, said first face being exposed to the exterior and said second face being exposed to the casing, and is provided with a retaining means, preferably a lug, which extends from the second face to the casing.

5. The system according to claim 2, in which said retaining means further comprise a lug extending from said wall in the direction of the second part of the wall of the slide, preferably under the slide.

6. The system according to claim 2, in which said support is a socket.

7. The system according to claim 6, in which said socket is configured for being fixed permanently or temporarily to a base, said socket being provided with a wall comprising a lower face and an upper face from which two guide rails extend, each extending in a longitudinal direction parallel to the upper face of said socket, each rail being a sectional bar provided with a first wall extending perpendicularly to said upper face of said socket, along the latter, and a second wall extending approximately perpendicularly from said first wall and parallel to said upper face of said socket, said socket being configured for housing the electronic monitoring device.

8. The system according to claim 7, in which each first wall of said two guide rails comprises retaining means, for example at least one lateral projection, preferably opposite one another, and said second part of the wall of said slide comprises a first lateral face and a second lateral face, with each lateral face comprising retaining means, for example at least one slot, said slots being located on either side of said second part of the wall of said slide and being configured for housing said lateral projections of said socket, when the casing of the electronic monitoring device is housed in the socket, preferably by forced fitting.

9. The system according to claim 7, in which said base may be a medicine box, a tablet container, a weekly dispenser, a bottle, a garment, a piece of furniture, or a sanitary fitting.

10. The system according to claim 7, in which said lower face of said socket comprises an adhesive, for example of the double-sided type, self-gripping means, or a repositionable pressure-sensitive silicone.

11. A use of a system for tracking a health-related behaviour according to any one of the preceding claims, for measuring a patient's compliance with a prescribed dosage.
